(19) United States
(12) Patent Application Publication
(10) Pub. No.: US 2016/0256583 A1
(43) Pub. Date: Sep. 8, 2016

(54) DEODORIZING FILTER

(71) Applicant: TOAGOSEI CO., LTD., Minato-ku (JP)

(72) Inventor: Yoshinao YAMADA, Nagoya-shi (JP)

(73) Assignee: TOAGOSEI CO., LTD., Minato-ku, Tokyo (JP)

(21) Appl. No.: 15/028,858

(22) PCT Filed: Aug. 28, 2014
(86) PCT No.: PCT/JP2014/072655

§ 371 Date: Apr. 12, 2016

(30) Foreign Application Priority Data
Oct. 17, 2013 (JP) 2013-216609

(51) Int. Cl. A61L 9/014; B01J 20/16; B01J 20/10; B01J 20/18; B01J 20/04; B01J 20/22; B01J 20/28; B01J 20/02; B01J 20/06

(52) U.S. Cl. CPC A61L 9/014; B01J 20/0292; B01J 20/16; B01J 20/103; B01J 20/06; B01J 20/043; B01J 20/22; B01J 20/28047; B01J 20/18; B01J 20/28028

(57) ABSTRACT

The deodorizing filter 1 of the present invention is provided with a deodorizing fiber layer 10 which contains a fiber 11 and a chemical adsorption type deodorant 13 bonded to a surface of the fiber 11, and a thickness of the deodorizing fiber layer 10 is 0.3 mm or more, a basis weight of the deodorizing fiber layer 10 is in a range from 30 to 100 g/m², and a permeability measured by fragile form method is in a range from 50 to 350 cm³/(cm²·s).

### TECHNICAL FIELD

The present ion relates to a deodorizing filter excellent in permeability and deodorizing performances to an unpleasant malodorous gas.

### BACKGROUND ART

In recent years, a demand for a comfortable life has further been increasing, and filters having various functions such as antimicrobial, antivirus, and deodorization, have heretofore been proposed. Among these, there are many proposals on a deodorizing filter having a deodorant which is to comply with respective malodorous components contained in a malodorous gas such as a stool odor, a putrid odor and a tobacco odor that a human body feels an unpleasant odor. For example, Patent Document I discloses a deodorant activated charcoal sheet obtained using an activated charcoal as a deodorant. Whereas the sheet has high permeability, the activated charcoal is a physical adsorption type deodorant, desorption of the malodorous component of which is reversible and a desorption rate of the same is rapid, so that a sufficient deodorization effect cannot be obtained and it is not sufficient for the use of a filter for adsorbing the malodorous component. Further, there is a problem that a gas containing the malodorous component is released again by a continuous use. Patent Document 2 discloses a deodorizing filter material in which two kinds of activated charcoals having different particle diameters are sandwiched between the nonwoven fabrics, and discloses a deodorizing filter. However, there is no detailed description about the deodorization effect, whereby it is uncertain whether a practical deodorization effect can be obtained or not. Moreover, the adsorbent is used in a large amount as 75 to 450 g/m²per a unit area of the filter, so that there is a fear that the adsorbent may be dropped.

Patent Document 3 discloses a photocatalyst-carried deodorization sheet in which titanium oxide as a photocatalyst is impregnated at one surface side and an activated charcoal is provided at the other surface side, and discloses a filter for air purification. Since a light is required to decompose the malodorous component using the photocatalyst, it is difficult to use the sheet in a dark place, whereby the use thereof is limited or a light source is required to be separately provided.

Patent Document 4 discloses a deodorizing wet nonwoven fabric including a fiber onto which a metal complex having an oxidation-reduction ability is carried and a fiber onto which an ion of a metal such as copper, cobalt and iron is carried. These components are considered to be a chemical adsorption type deodorant, and high deodorizing performance could be obtained depending on the method of use. But there is no detailed description about a specific deodorization effect. It is uncertain whether the practical deodorization effect can be obtained or not. In addition, there description about permeability of a filter.

Patent Document 5 discloses a deodorizing material in which either of an oxide, a hydroxide or a complex oxide, of manganese, cobalt, copper or zinc is carried onto, and the material is for deodorizing a complex odor containing a sulfur-based odor by a toilet odor, and an odor at the time of defecation as a target. Since these compounds are chemical adsorption type deodorants, there is a possibility that high deodorizing performance can be obtained. In the deodorizing material, a sufficient deodorization effect cannot be obtained by using the chemical adsorption type deodorant alone, and a physical adsorption type deodorant must be used in combination with the chemical adsorption type deodorant.

Patent Document 6 discloses a wet nonwoven fabric in which a deodorant is attached to a nonwoven fabric a fiber diameter and a fiber length of which are controlled. However, there is no description about the deodorization effect other than the gas containing a malodorous component of ammonia, so that it is uncertain whether the practical level of a deodorization effect can be obtained or not as a deodorizing filter.

Patent Document 7 discloses a laminated sheet in which a layer including a substance which has a function of adsorbing a material having an odor, and an air-permeable sheet layer including a photocatalyst which decomposes the substance having an odor are laminated and integrated. Patent Document 8 discloses a deodorizing and antibacterial sheet containing an adsorbent and a photocatalyst. A time until ammonia and acetaldehyde which are components to be deodorized are decomposed is 10 minutes or longer, and there is no description about the deodorizing performance with a time shorter than the above. it is uncertain whether the practical level of a deodorization effect can be obtained or not.

Further, Patent Document 9 discloses a pleated type air filter material including a nonwoven fabric sheet for deodorization having high permeability and a dust filter body. However, it is uncertain whether a sufficient deodorization effect can be obtained or not against the malodorous gas other than ammonia and acetaldehyde.

On the other hand, chemical adsorption type deodorants which can exhibit high deodorizing performance with a little amount are disclosed in Patent Documents 10, 11 and 12. The chemical adsorption type deodorant has an effect of deodorization by a reaction with a malodorous compontent within a short period of time. However, a state of the bad odor which is an object of the deodorizing filter is generally a gas, and the deodorant and the malodorous gas are instantaneously contacted. As long as the nonwoven fabric onto which a deodorant is carried has permeability, the malodorous gas which passes through the fabric without contacting with the deodorant necessarily exists. A deodorizing filter which can deodorize the had odor substantially no odor has not yet been realized. On the other hand, a demand for comfortability has been increasing in recent years, and a deodorizing filter having high deodorizing performance is desired which adsorbs the malodorous gas with good efficiency and does not generate an unpleasant feeling,

### PRIOR TECHNICAL LITERATURE

### Patent Literature

[Patent Document 1] JP-A 2005-349570

[Patent Document 2]JP-A 2003-320209

[Patent Document 3]JP-A 2002-17836

[Patent Document 4] JP-A Sho 62-7000

[Patent Document 5] JP-A 2004-129840

[Patent Document 6] JP-A 2012-92466

[Patent Document 7]JP-A 2008-104556

[Patent Document 8]JP-A 2008-104557

[Patent Document 9] JP-A 2003-299919

[Patent Document 10] JP-A 2000-279500

[Patent Document 11] JP-A 2002-200149

[Patent Document 12] JP-A 2011-104274

### SUMMARY OF THE INVENTION

### Problems that the Invention is to Solve

An object of the present invention is to provide a deodorizing filter excellent in permeability and deodorizing performances to an unpleasant malodorous gas.

### Means to Solve the Problems

The present invention is directed to a deodorizing filter which has a deodorizing fiber layer including a fiber and a chemical adsorption type deodorant joined to a surface of the fiber, and is a deodorizing filter characterized in that a thickness of the deodorizing fiber layer is 0.3 mm or more, a basis weight of the deodorizing fiber layer is in a range from 30 to 100 g/m², and permeability (permeability from one surface side to the other surface side) of the deodorizing filter is in a range from 50 to 350 cm³/(cm²·s),

In the present invention, a substance which is a cause of bad smell is called to as “malodorous component”, and a gas containing the malodorous component is called to as “a malodorous gas”. A unit “ppm” with regard to the gas concentration is “ppm by volume”. Further, a “permeability” is permeability measured by fragile form method accordine to JIS L 1096:2010.

### EFFECT OF THE INVENTION

The deodorizing filter of the present invention has sufficient permeability from one surface side to the other surface side, and has excellent deodorizing performances to an unpleasant malodorous gas. In particular, under a stream of the malodorous gas in which permeability is in a range from 50 to 350 cm³/(cm²·s), deodorization can be efficiently carried out by an instantaneous contact of the malodorous component and the deodorizing filter. Accordingly, when the deodorizing filter of the present invention is used as a filter for adsorbing a malodorous component contained in the malodorous gas such as a stool odor, a putrid odor, and a tobacco odor, the malodorous component in the atmosphere can be reduced.

The deodorizing filter of the present invention is useful as a filter for a mask, or a filter for an air cleaner, an air conditioner and the like to avoid bad smell generated in medical, caregiving and excrementitious fields, a wastewater treatment plant, a refuse treatment plant (an incineration plant), a fertilizer factory, a chemical factory, or the like; an animal smell, a stool odor, a putrid odor (including a bad odor from a pet or an article for pets) generated in a livestock farm, a fishing port, an animal-related institution, or the like; bad smell from a foot stepping mat, shoe insoles, a shoe cupboard, a trash can, a toilet, or the like.

### BRIEF DESCRIPTION OF DRAWINGS

FIG. 1 is a schematic view showing one example of cross-section structure of the deodorizing filter of the present invention,

FIG. 2 is a schematic view showing other example of cross-section structure of the deodorizing filter of the present invention.

FIG. 3 is a schematic view showing other example of cross-section structure of the deodorizing filter of the present invention.

FIG. 4 is a graph showing evaluation results of persistence of the deodorant tests in Example 12 and Comparative example 7.

### EMBODIMENTS FOR CARRYING OUT THE INVENTION

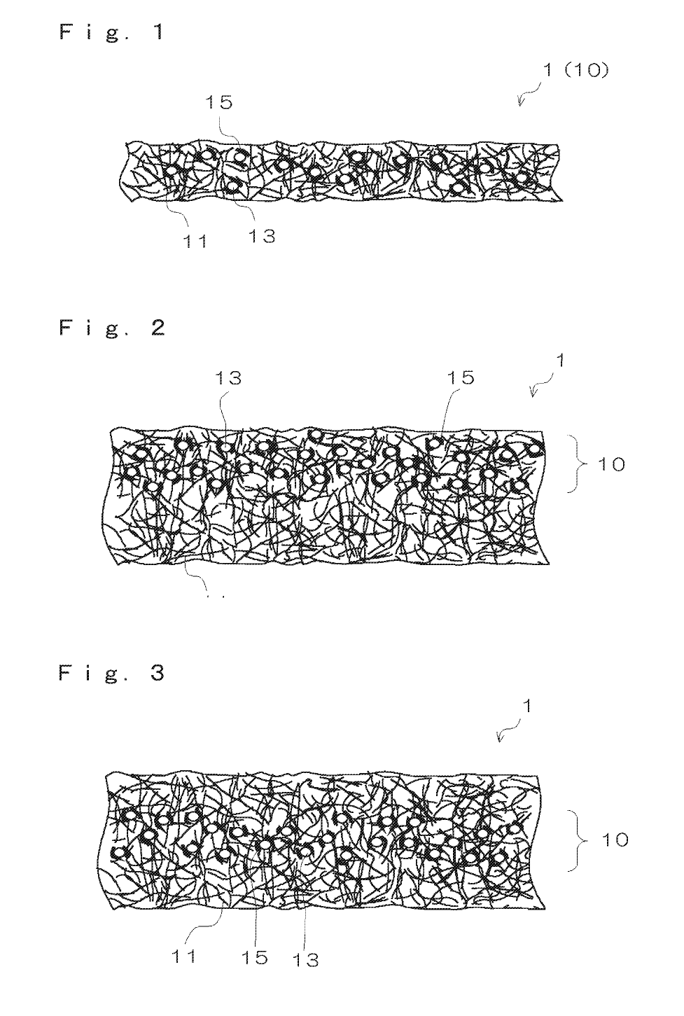

The deodorizing filter of the present invention is a filter that has a deodorizing fiber layer including a fiber and a chemical adsorption type deodorant joined to a surface of the fiber, and has permeability from one surface side to the other urface side of the filter sandwiching the deodorizing fiber layer. And the deodorizing filter of the present invention may be a deodorizing filter 1 having a cross-section structure in which the whole portion thereof is consisting of a deodorizing fiber layer 10 (see FIG. 1), or a deodorizing filter 1 having a cross-section structure in which a part thereof is a deodorizing fiber layer 10 (see FIGS. 2 and 3). The deodorizing fiber layer may be either a single layered structure or a multi-layered structure. The deodorizing filter of the present invention may be used to adapt to the objective size or shape (a plane structure, a cubic structure such as pleat).

The deodorizing fiber layer constituting the deodorizing filter of the present invention is preferably a fiber aggregation containing at least one selected from a composite fiber in which a chemical adsorption type deodorant is so buried at a surface of the base portion of the fiber that the deodorant is exposed and a composite fiber in which a chemical adsorption type deodorant is joined onto a surface of the fiber through an adhesive layer. The fiber aggregation may contain a fiber having no chemical adsorption type deodorant. An average diameter of the fiber such as a composite fiber contained in the fiber aggregation is generally in a range from 5 to 30 μm, and preferably from 10 to 25 μm.

A base material constituting the deodorizing fiber layer (or a deodorizing filter) may consist of either a woven fabric or a nonwoven fabric. The base material preferably consists of a nonwoven fabric since setting of a desired thickness is easy, a production cost is inexpensive and permeability can be easily controlled.

Examples of a resin constituting the fiber contained in the nonwoven fabric include a polyester, a polyethylene, a polypropylene, a poly vinyl chloride, a polyacrylic acid, a polyamide, a poly vinyl alcohol, a polyurethane, a poly vinyl ester, a poly inethacrylic acid ester, a rayon, and the like. Among these, a polyethylene, a polypropylene, a polyester and a rayon are preferable since adhesiveness between the chemical adsorption type deodorant and a binder resin and permeability can be sufficiently obtained in an embodiment where the chemical adsorption type deodorant is joined onto a surface of the fiber through an adhesive layer including the binder resin. The nonwoven fabric may be a nonwoven fabric having a fiber containing only one type of a resin or a nonwoven fabric having a plural types of resin fibers. The nonwoven fabric is preferably a nonwoven fabric interlacing by a needle-punching method, a water-jet interlacing method (spunlace method), or the like, a nonwoven fabric produced by a thermal bonding method, and a nonwoven fabric produced by a spun bonding method.

As a deodorant for the malodorous gas, a deodorant for adsorbing the malodorous component by physical adsorption such as an activated charcoal and a deodorant for decomposing the malodorous component at a time of contact such as a photocatalyst are generally used in addition to the deodorant in which the malodorous component is adsorbed by chemical adsorption or a chemical bond is formed with the malodorous component such as the chemical adsorption type deodorant of the present invention. However, whenit is used for a filter to ventilate the malodorous gas, it is necessary to adsorb the malodorous component within a short time during which the malodorous gas passes, so that a sufficient deodorization effect cannot be obtained in the physical adsorption type deodorant in which the malodorous gas is released again by continuous use or the decomposition type deodorant in which the component is decomposed by irradiating light. As the deodorant to be used for the deodorizing fiber layer constituting the deodorizing filter, a chemical adsorption type deodorant is optimum since it can adsorb the malodorous component within a short time, exhibits a sufficient deodorization effect at a time of passing through the deodorizing fiber layer, and has a rapid deodorizing speed and a large deodorization capacity. A form of the chemical bond in the chemical adsorption type deodorant is not particularly limited, and depends on a functional group contained in the chemical adsorption type deodorant, a functional group contained in the malodorous component, or the like in some cases.

The malodorous component which is an object of the chemical adsorption type deodorant to be deodorized are specifically a basic compound such as ammonia and an amine; an acidic compound such as acetic acid and isovaleric acid; an aldehyde such as formaldehyde, acetaldehyde and nonenal; a sulfur compound such as hydrogen sulfide and methyl mercaptan; and the like.

Examples of the chemical adsorption type deodorant to these malodorous components include an inorganic-based chemical adsorption type deodorant and an organic-based chemical adsorption type deodorant. Specific examples of the inorganic-based chemical adsorption type deodorant include a tetravalent metal phosphate, a zeolite, an amorphous composite oxide, a composite material containing at least one atom selected from Ag, Cu, Zn and Mn, a zirconium compound selected from a hydrated zirconium oxide and a zirconium oxide, a hydrotalcite-based compound, an amorphous active pound, and the like. Examples of the organic-based chemical adsorption type deodorant include an amine compound, and the like. A deodorant excellent in safety and difficultly deteriorated is preferably an inorganic-based chemical adsorption type deodorant which is insoluble or hardly soluble in water.

The chemical adsorption type deodorant may be used singly or in combination of two or more ypes thereof. When a plural number of the chemical adsorption type deodorants different in objects to be deodorized (malodorous components), synergistic effects can be obtained in some cases. For example, to a stool odor or putrid odor (an odor of garbage or the like) containing ammonia, trimethylamine, hydrogen sulfide, methyl mercaptan, dimethyl disulfide, or the like, a combination of a chemical adsorption type deodorant for a basic gas and a chemical adsorption type deodorant for a sulfur-based gas is suitable. For example, to a body odor such as sweat smell,containing acetic acid, isovaleric acid, or the like, a combination of a chemical adsorption type deodorant for a basic gas and a chemical adsorption type deodorant for an acidic gas is suitable. In addition, to a tobacco odor containing acetaldehyde, acetic acid, or the like, a combination of a chemical adsorption type deodorant for a basic gas, a chemical adsorption type deodorant for an acidic gas and a chemical adsorption type deodorant for an aldehyde gas is suitable. A ratio of the amounts to be used when two or more types of the chemical adsorption type deodorants are used in combination is preferably selected depending on deodorizing performances such as deodorization capacity and deodorizing speed of the chemical adsorption type deodorant to be used, and a gas concentration of an environment to be objected (a concentration of the malodorous component). In a case where two types of chemical adsorption type deodorants are used to deodorize a malodorous gas containing a plural kind of malodorous components, for example, an approximate mass ratio thereof for obtaining a sufficient deodorization effect is 20:80 to 80:20. In addition, the chemical adsorption type deodorant according o the present invention and a physical adsorption type deodorant such as an activated charcoal may be used in combination. A deodorization capacity means an amount (mL) of the malodorous component in the standard state capable of deodorizing with 1 g of the chemical adsorption type deodorant, and the larger value thereof gives a deodorization effect persistence of the deodorizing filter.

Next,the chemical adsorption type deodorant to be used in the present invention is described.

### (A) Tetravalent Metal Phosphate

The tetravalent metal phosphate is preferably a compound represented by the following general formula (1). The compound is insoluble or hardly soluble in water, and excellent in a deodorization effect to a basic gas.

HₐM_{b}(PO₄)_{c}.nH₂O (1)

(In the formula, M is a tetravalent metal atom, a, h and c are each an integer satisfying a formula: a+4b=3c, and n is 0 or a positive integer.)

Examples of M in the general formula (1) include Zr, Hf, Ti, Sn, and the like.

Preferable specific examples of the tetravalent metal phosphate include zirconium phosphate (Zr(HPO₄)₂.H₂O), hafnium phosphate, titanium phosphate, tin phosphate, and the like. There are crystalline substances having various crystal systems such as a type crystal, β type crystal, and y type crystal, and an amorphous substance in these compounds, either of which is preferably used.

### (B) Amine Compound

The amine compound is preferably a hydrazine-based compound or an aminoguanidine salt. Since these compounds react with an aldehyde-based gas, they are excellent in deodorization effect to the aldehyde-based gas. Examples of the hydrazine-based compound include adipic acid dihydrazide, carhohydrazide, succinic dihydrazide, and oxalic dihydrazide. Examples of the aminoguanidine salt include aminoguanidine hydrochloride, aminoguanidine sulfate, aminoguanidine bicarbonate, and the like. These amine compounds may constitute a deodorant carried on a carrier. A material for the carrier in this case is generally an inorganic compound, and specific example thereof includes a zeolite, an amorphous composite oxide mentioned below, a silica gel, and the like. Since the zeolite and the amorphous composite oxide each have a deodorization effect to a basic gas, when these materials are used as the carrier, it is effective to both of the aldehyde-based gas and the basic gas.

### (C) Zeolite

The zeolite is preferably a synthetic zeolite. The zeolite is insoluble or hardly soluble in water, and is excellent in deodorization effect to a basic gas. There are various structures of the zeolite such as A type, X type, Y type, a type, β type, ZSM-5, and amorphous, and either of the conventionally known zeolites can be used.

### (D) Amorphous Composite Oxide

The amorphous composite oxide is a compound other than the above zeolite. The composite oxide is preferably an amorphous composite oxide constituted by at least two types selected from Al₂O₃, SiO MgO, CaO, SrO, BaO, ZnO, ZrO₂, TiO₂, WO₂, CeO₂, Li₂O, Na₂O, K₂O, or the like. The composite oxide is insoluble or hardly soluble in water, and is excellent in deodorization effect to a basic gas. An amorphous composite oxide represented by X₂O—Al₂O₃—SiO₂ (X is at least one alkali metal atom selected from Na, K and Li) is particularly preferred since it is excellent in deodorizing performances. Being amorphous means no clear diffraction signals based on crystal face can be recognized when X-ray powder diffraction analysis is carried out, more specifically, high kurtosis (the so-called sharp) signal peak is not substantially appeared in the X-ray diffraction chart in which a diffraction angle is plotted at the transverse axis and a diffraction signal intensity is plotted at the vertical axis.

(E) Composite Material Containing at Least One Atom Selected from Ag, Cu, Zn and Mn

The composite material s a composite material insoluble or hardly soluble in water, and is excellent in deodorization effect to a sulfur-based gas. The composite material is a composite material consisting of at least one type selected from at least one atom selected from Ag, Cu, Zn and Mn and a compound containing he atom, and the other material(s). The compound containing at least one atom selected from Ag, Cu, Zn and Mn is preferably an oxide, a hydroxide, a salt of an inorganic acid such as phosphoric acid and sulfuric acid, and a salt of an organic acid such as acetic acid, oxalic acid and acrylic acid. Accordingly, as the deodorant (E), a composite material insoluble in water in which at least one metal selected from Ag, Cu, Zn and Mn or the compound is/are carried on a carrier consisting of an inorganic compound that is the other material can be used. An inorganic compound preferred as the carrier is silica, a tetravalent metal phosphate, or a zeolite. Since the tetravalent metal phosphate and zeolite have deodorization effects to a basic gas, when the tetravalent metal phosphate and zeolite are used as the carrier, it is effective to both of a sulfur-based gas and the basic gas.

### (F) Zirconium Compound

The zirconium compound is hydrated zirconium oxide or zirconium oxide. The zirconium compound is preferably an amorphous compound. These compounds are insoluble or hardly soluble in water, and excellent in deodorization effect to an acidic gas. The hydrated zirconium oxide is a compound which has the same meanings as zirconium oxyhydroxide, zirconium hydroxide, hydrous zirconium oxide or zirconium oxide hydrate.

### (G) Hydrotalcite-Based Compound

The hydrotalcite-based compound is a compound having a hydrotalcite structure, and is preferably represented by the following general formula (2). The compound is insoluble or hardly soluble in water, and excellent in deodorization effect to an acidic gas.

M^{1(1-x)}M²ₓ(OH)₂Aⁿ⁻_{(x/n)}.mH₂O (2)

(In the formula, M¹ a divalent metal atom, M² is a trivalent metal atom, x is a number larger than 0 and 0.5 or less, Aⁿ⁻ is an n-valent anion such as a carbonic acid ion, and a sulfuric acid ion, and m is a positive integer.)

Examples of the hydrotalcite-based compound include magnesium-aluminum hydrotalcite, zinc-aluminum hydrotalcite, and the like. Among these, magnesium-aluminum hydrotalcite is particularly preferred since it shows more excellent deodorization effect to an acidic gas. A calcined product of the hydrotalcite, i.e., a compound obtained by calcining the hydrotalcite compound at a temperature of about 500° C. or higher to remove a carboxylate or a hydroxyl group is also included in the hydrotalcite-based compound.

### (H) Amorphous Active Oxide

The amorphous active oxide is a compound not containing the amorphous composite oxide, The amorphous active oxide is preferably insoluble or hardly soluble in water, and excellent in deodorization effect to an acidic gas or a sulfur-based gas. Specific xamples of the amorphous active oxide include Al₂O₃, SiO₂, MgO, CaO, SrO, BaO, ZnO, CuO, MnO, ZrO₂, TiO₂, WO₂, CeO₂, and the like. In addition, a surface-treated active oxide may be used. Specific examples of the surface-treated product include an active oxide surface-treated by an organopolysloxane, and an active oxide in which the surface is coated by an oxide or a hydroxide of aluminum, silicon, zirconium or tin. An amorphous active oxide subjected to surface treatment with an organic-based material such as an organopolysiloxane is preferred than an amorphous active oxide subjected to surface treatment with an inorganic-based material since the former gives higher deodorizing performances.

A shape of the chemical adsorption type deodorant according to the present invention is not particularly limited, With regard to the size of the chemical adsorption type deodorant, when it is in granular, a median diameter measured by a laser diffraction particle size distribution analyzer is preferably in a range from 0.05 to 100 μm, more preferably from 0.1 to 50 μm, and further preferably from 0.2 to 30 μm from the viewpoint of the deodorization efficiency. If the chemical adsorption type deodorant is too large, a surface area of the exposed chemical adsorption type deodorant per a unit mass is small, so that there is a case where a sufficient deodorization effect cannot be obtained or there is a case where a sufficient permeability cannot be obtained when a desired basis weight has been set.

An excellent deodorization effect can be obtained as the efficiency of contacting the chemical adsorption type deodorant with the malodorous component becomes higher, so that a specific surface area is preferably in a range from 10 to 800 m²/g, and more preferably from 30 to 600 m²/g. The specific surface area can be measured by BET method which calculates from a nitrogen adsorption amount.

In the deodorizing fiber layer constituting the deodorizing filter of the present invention, a content of the chemical adsorption type deodorant per unit area is preferably larger. However, as the content s larger, permeability of the deodorizing filter is lowered and a cost thereof is increased so that the content is generally determined in consideration of the above. The content of one kind of the chemical adsorption type deodorant in the deodorizing fiber layer is preferably /m² or more, more preferably 3 g/m² or more, and further preferably 5 g/m² or more. In addition, the total content when two or more kinds of the chemical adsorption type deodorants are contained is preferably 2 g/m² or more, more preferably 6 g/m² or more, and further preferably 10 g/m² or more.

In the present invention, a preferred embodiment of the deodorizing fiber layer which gives an excellent deodorization effect is that a content ratio of the chemical adsorption type deodorant is preferably set in a range from 2 to 60 parts by mass, more preferably from 5 to 50 parts by mass, and further preferably from 10 to 40 parts by mass based on 100 parts by mass of a mass of fibers constituting the deodorizing fiber layer.

The deodorizing fiber layer may have, as mentioned above, an embodiment in which the chemical adsorption type deodorant is embedded at a surface of the fiber, or an embodiment in which the fiber and the chemical adsorption type deodorant are joined through an adhesive layer. In the latter case, examples of a constitutional material (a binder resin) for the adhesive layer include a natural resin, a natural resin derivative, phenol resin, a xylene resin, a urea resin, a melamine resin, a ketone resin, a coumaron-indene resin, a petroleum resin, a terpene resin cyclized rubber, a chlorinated rubber, an alkyd resin, a polyamide resin, a polyvinyl chloride resin, an acrylic resin, a vinyl chloride-acetic acid vinyl copolymer resin, a polyester resin, a polyvinyl acetate, a polyvinyl alcohol, a polyvinyl butyral, a chlorinated polypropylene, a styrene resin, an epoxy resin, a urethane-based resin, a cellulose derivative, and the like. Among these, an acrylic-based resin, a urethane-based resin, a polyester esin and a polyvinyl alcohol are preferred. The binder resin may he used singly or in combination of two or more types thereof.

In the deodorizing filter having the adhesive layer, if an amount of the chemical adsorption type deodorant per a unit area in the deodorizing fiber layer is increased to improve deodorization effect, an amount of the binder resin to be used for joining the chemical adsorption type deodorant is also generally increased, and buried between fibers constituting the deodorizing fiber layer whereby lowering permeability of the deodorizing filter. Further, an amount of the chemical adsorption type deodorant buried in the binder resin is increased so that the deodorant cannot be contacted with the malodorous component contained in the malodorous gas, whereby a deodorization effect which can be expected to the deodorant accompanied by an increase in the content thereof cannot be obtained. Thus, a thickness and a basis weight of the deodorizing fiber layer are within the specific ranges, and permeability of the deodorizing filter is also within the specific range for the purpose of sufficiently obtaining deodorization effect of the chemical adsorption type deodorant without lowering permeability in the deodorizing filter of the present invention.

With regard to the thickness of the deodorizing fiber layer in the deodorizing filter of the present invention, a sufficient deodorization effect can be obtained when it is 0.3 mm or more. The thickness is preferably in a range from 0.3 to 1.5 mm, and more preferably from 0.5 to 1.2 mm from the viewpoint of practicability in the field mentioned later. The thickness of the deodorizing fiber layer is the same as that for the multi-layered deodorizing fiber layer mentioned later as well. A basis weight of the deodorizing fiber layer is in a range from 30 to 100 g/m² since a sufficient deodorization effect and permeability can be obtained. The basis weight is preferably from 35 to 90 g/m², and more preferably from 40 to 85 g/m². The basis weight of the deodorizing fiber layer is the same as that for the multi-layered deodorizing fiber layer mentioned later as well. When the thickness of the deodorizing fiber layer is in a range from 0.3 to 1.5 mm and the basis weight is in a range from 30 to 100 g/m², while it has high permeability, the malodorous component(s) is/are sufficiently adsorbed by the chemical adsorption type deodorant, and excellent deodorizing performances to the malodorous gas can be obtained.

A balance between the thickness of the deodorizing fiber layer and the basis weight is important for the purpose of giving high permeability to the deodorizing filter and obtaining high deodorizing performances, and such a balance could be firstly accomplished by the present invention.

A permeability of the deodorizing fiber layer is preferably in a range from 50 to 350 cm³/(cm²·s), more preferably from 100 to 350 cm³/(cm²·s), and further preferably from 170 to 300 cm³/(cm²·s) since a deodorization effect with good efficiency can be obtained.

In the present invention, if the thickness of the deodorizing fiber layer is less than 0.3 mm, a sufficient deodorization effect cannot be obtained.

If the basis weight of the deodorizing fiber layer is less than 30 g/m², permeability of the deodorizing fiber layer becomes too high, so that the malodorous component in the malodorous gas does not contact with the chemical adsorption type deodorant and almost all the malodorous gas passes through the deodorizing fiber layer, whereby the deodorization effect is lowered. On the other hand, if the basis weight exceeds 100 g/m², permeability of the deodorizing fiber layer is markedly lowered, and a gas does not smoothly flow from the one side surface to the other side surface of the deodorizing filter.

The deodorizing filter of the present invention may have a cross-section structure shown in FIG. 1, 2 or 3. The deodorizing fiber layer may be a single layer formed from a fiber aggregation which includes a composite fiber containing one or two or more types of the chemical adsorption type deodorant, or may he a plural layer using two or more of the fiber aggregation. The deodorizing fiber layer may be a layer formed from a fiber aggregation which includes a composite fiber containing a chemical adsorption type deodorant, and a (at least one type of) composite fiber(s) containing the other (at least one type of) chemical adsorption type deodorant(s). Whereas it is not shown in the drawing, he deodorizing fiber layer 10 may be a multi-layered deodorizing fiber layer consisting of a fiber layer containing a chemical adsorption type deodorant, and a fiber layer containing the other chemical adsorption type deodorant. The deodorizing fiber having a cross-section structure a part of which is the deodorizing fiber layer 10 may, as shown in FIGS. 2 and 3, be a laminated type deodorizing filter consisting of a deodorizing fiber layer 10 and a fiber layer (containing a fiber which is the same as or different from the fiber constituting the deodorizing fiber layer 10, which is a fiber layer having permeability from one surface side to the other surface side, and hereinafter referred to as “the other fiber layer”) having a function other than the deodorization (dustproof, protection of the deodorizing fiber layer, etc.), depending on necessity. The other fiber layer may consist of either a woven fabric or a nonwoven fabric. A basis weight of the other fiber layer is not particularly limited. A permeability of the other fiber layer is preferably higher than that of the deodorizing fiber layer 10. A number of the other fiber layer may be 1 or 2 or more. The thickness of the other fiber layer is not particularly limited.

With regard to the permeability of the deodorizing filter of the present invention, when the permeability is low, contacting efficiency of the malodorous component contained in the malodorous gas and the chemical adsorption type deodorant contained in the deodorizing fiber layer tends to be high, so that a high deodorization effect tends to be obtained but as the performance of the filter, the permeability is preferably high. However, if the permeability is too high, the malodorous gas passes through voids of the deodorizing fiber layer, and the chemical adsorption type deodorant cannot adsorb the malodorous component with good efficiency whereby the deodorizing performance is lowered. Accordingly, the permeability of the deodorizing filter to develop the high deodorization effect is in a range from 50 to 350 cm³/(cm²·s), more preferably from 100 to 350 cm³/(cm²·s), and further preferably from 170 to 300 cm³/(cm²·s).

The deodorizing filter of the present invention can be produced by the various methods to form the constitution, which are exemplified by the following.
(1) A method in which a deodorant composition containing a chemical adsorption type deodorant and a binder resin is subjected to coating (dipping, spraying, padding, or the like) to the whole of a woven fabric or a nonwoven fabric consisting of a fiber containing no chemical adsorption type deodorant, drying is conducted to adhere the chemical adsorption type deodorant onto a surface of the fiber constituting the woven fabric or the nonwoven fabric, and a deodorizing filter consisting substantially of a deodorizing fiber layer is produced.
(2) A method in which a deodorant composition containing a chemical adsorption type deodorant and a binder resin is subjected to coating (dipping, spraying, padding, or the like) to the whole of a woven fabric or a nonwoven fabric consisting of a fiber containing no chemical adsorption type deodorant, drying is conducted to adhere the chemical adsorption type deodorant onto a surface of the fiber constituting the woven fabric or the nonwoven fabric, a sheet for a deodorizing fiber layer is prepared, the sheet and a woven fabric or nonwoven fabric that includes the other fiber containing no chemical adsorption type deodorant are bonded by using a binder resin, interlacing treatment, or the like, and a multi-layered deodorizing filter consisting of a deodorizing fiber layer and the other fiber layer is produced.
(3) A method in which a deodorant composition containing a chemical adsorption type deodorant and a binder resin is subjected to coating ((dipping, spraying, padding, or the like) to a part (one-face side surface layer or an inside alone) in a cross-sectional direction of a woven fabric or a nonwoven fabric including a fiber containing no chemical adsorption type deodorant, drying is conducted to adhere the chemical adsorption type deodorant onto a surface of the fiber constituting the woven fabric or the nonwoven fabric, and a deodorizing filter consisting of a deodorizing fiber layer and a fiber layer containing no chemical adsorption type deodorant is produced.
(4) A method in which a woven fabric or nonwoven fabric consisting of a composite fiber in which a chemical adsorption type deodorant is buried on a surface of a base portion of the fiber so that the deodorant is exposed is used or subjected, if necessary, to an interlacing treatment (a needle-punching method, or the like), and a deodorizing filter consisting substantially of a deodorizing fiber layer is produced.
(5) A method in which a woven fabric or nonwoven fabric consisting of a composite fiber in which a chemical adsorption type deodorant is buried on a surface of a base portion of the fiber so that the deodorant is exposed and a woven fabric or nonwoven fabric that includes the other fiber containing no chemical adsorption type deodorant are bonded by using a binder resin, interlacing treatment, or the like, and a multi-layered deodorizing filter consisting of a deodorizing fiber layer and the other fiber layer is produced.
(6) A method in which a woven fabric or a nonwoven fabric including a fiber containing no chemical adsorption type deodorant is subjected to a heat treatment or a chemical treatment, in a state of contacting the chemical adsorption type deodorant, to fix the chemical adsorption type deodorant to a surface of the fiber and a deodorizing filter consisting substantially of a deodorizing fiber layer is produced.

In the present invention, the developing processing method (1) is particularly preferred.

The chemical adsorption type deodorant and the binder resin contained in the deodorant composition for the method (1) are as already mentioned above. In particular, a median diameter of the chemical adsorption type deodorant contained in the deodorant composition is preferably in a range from 0.05 to 100 μm since smooth developing processing can be carried out. A chemical adsorption type deodorant having small median diameter is preferable since a surface area per a unit mass becomes larger, deodorization efficiency is improved, developing processing can be easily carried out, and dropping after the processing is hardly occurred. If a chemical adsorption type deodorant having a median diameter of less than 0.05 μm is used, there causes inconveniences that the chemical adsorption type deodorant is buried at an inside of the adhesive layer and not exposed, and that the chemical adsorption type deodorant causes secondary flocculation at a time of the developing process and undissolved lumps are formed at a surface of the woven fabric or the nonwoven fabric, which drop after the processing. A median diameter of the chemical adsorption type deodorant is more preferably in a range from 0.1 to 50 μm, and further preferably from 0.2 to 30 μm,

Depending on types of the chemical adsorption type deodorants, a deodorization effect is sometimes lowered by coexisting these proximately in the deodorizing fiber layer. Therefore, a plural types of the chemical adsorption type deodorants are to be fixed, it is necessary to select a method in which a deodorant composition containing a plural types of the chemical adsorption type deodorant is prepared and the composition is used for developing processing as it is or a method in which a plural types of deodorant compositions each containing one kind of the chemical adsorption type deodorant are prepared and developing processing is carried out repeatedly by using these compositions separately, In addition, the developing processing may be carried out using a deodorant composition containing a chemical adsorption type deodorant and a physical adsorption type deodorant such as an activated charcoal.

In a case where a deodorant composition containing a binder resin and a chemical adsorption type deodorant is used, higher ratio of the binder resin to the chemical adsorption type deodorant is preferred from the viewpoint that a fixing force of the chemical adsorption type deodorant is heightened and dropping of the chemical adsorption type deodorant is suppressed. On the other hand, when a ratio of the binder resin is low, the chemical adsorption type deodorant can be easily exposed, and as a result, the chemical adsorption type deodorant is easily contacted with the malodorous component contained in the malodorous gas whereby an excellent deodorization effect can be obtained. Accordingly, to expose the chemical adsorption type deodorant with good efficiency and to obtain an excellent deodorization effect, a content ratio of the binder resin and the chemical adsorption type deodorant is preferably in a range from 10% to 90% by mass and 10% to 90% by mass, and more preferably 20% to 50% by mass and 50% to 80% by mass, respectively, based on 100% by mass of a total of the binder resin and the chemical adsorption type deodorant.

When an additive is added to the deodorant composition depending on types of the binder resin, a function other than the deodorizing performance can be provided or improvement of the developing processing property, or he like can be done. Examples of the additive include a dispersant, a defoaming agent, a viscosity modifier, a surfactant, a pigment, a dye, a fragrant, an antimicrobial agent, an antiviral agent, an antiallergenic agent, and the like. A formulation amount of the additive is required to be optionally selected so as not to lower the deodorization effect of the chemical adsorption type deodorant or not affect to the permeability of the deodorization nonwoven fabric.

When the deodorant composition is prepared, a general dispersing method of inorganic powder or the like can be applied. For example, an additive for a binder resin such as a dispersing agent is added to an emulsion of the binder resin, a chemical adsorption type deodorant is further added, and the mixture is stirred using a sand mill, a dspermill, a ball mill or the like, to mix and disperse the deodorant. In the case of the preparation method, as a solid concentration of the chemical adsorption type deodorant in the deodorant composition is higher, a viscosity of the binder composition is increased and handling thereof becomes difficult but drying of the coated film can be carried out efficiently. Therefore, a solid concentration of the chemical adsorption type deodorant in the deodorant composition is preferably in a range from 5% to 30% by mass. For the purpose of adjusting the viscosity of the deodorant composition, a viscosity modifier or the like may be used within a range which does not exert an effect to the deodorizing performance.

A developing processing method of the deodorant composition containing the chemical adsorption type deodorant to a base material woven fabric or a nonwoven fabric) is as mentioned above. Example of the dipping method includes a room temperature standing method, a heating and stirring method, and the like. Example of the padding method includes a pad drying method, a pad steam method, and the like. When the obtained base material attached with a film is dried to optionally remove a medium of the deodorant composition, the binder resin exhibits the function whereby the chemical adsorption type deodorant is adhered to the surface of the fiber constituting the base material. A drying temperature at this time is not particularly limited. When the deodorant composition is, for example, an emulsion composition,it is preferably in a range from 50° C. to 150° C. or so, and more preferably from 80° C. to 130° C. or so. A drying time varies depending on the drying temperature, but is preferably in a range from 2 minutes to 12 hours, and more preferably from 5 minutes to 2 hours. When the drying is conducted under such conditions, the chemical adsorption type deodorant can be efficiently fixed to the surface of the fiber constituting the base material while exposing.

When the deodorizing filter of the present invention is produced using the deodorant composition, it is preferred to use,as a base material, a nonwoven fabric produced by a needle-pun ing method, a nonwoven fabric produced by a thermal bonding method or a nonwoven fabric produced by a spun bonding method for the purpose of joining the chemical adsorption type deodorant uniformly to the surface of the fiber constituting the base material and making setting of permeability and thickness easy.

A deodorizing filter having a multi-layered deodorizing fiber layer can be produced by subjecting to coating and drying of the deodorant composition to a plural number of respective base materials, and laminating and integrating these. In this case, different chemical adsorption type deodorants may be applied to the respective base materials.

### EXAMPLES

Hereinafter, the present invention is specifically described using Examples. The present invention is not limited to the Examples. In the following description, “part(s)” and “%” are based on mass unless otherwise indicated.

A median diameter of a chemical adsorption type deodorant was measured with a volume standard using a laser diffraction particle size distribution, A permeability of a deodorizing filter was measured by fragile form method regulated in JIS L 1096:2010. The unit is cm³/(cm²·s). A thickness of the deodorizing filter was measured by a thickness gauge “PEACOCK No.25” (Trade Name) manufactured by OZAKI MFG. CO., Ltd., according to the method regulated in ES L 1096:2010. The unit is mm. A basis weight of the deodorizing filter was measured by the method regulated in JIS L 1096:2010 and expressed by a mass per 1 m² (g/m²), in the standard state.

A deodorization test was carried out in which a malodorous gas provisionally prepared by containing a predetermined concentration of a malodorous component(s) is passed from one surface side to the other surface side of the deodorizing filter, Specifically, while sucking the malodorous gas contained in the bag using a gas sampling pump “MODEL GV-100” (Type name) manufactured by GASTEC CORPORATION, it is passed through the deodorizing filter having an area of 5 cm² at a path, a concentration of the malodorous component in the passing gas was measured by a gas detecting tube.

As the malodorous gas, a gas containing ammonia (40 ppm), acetic acid (1.9 ppm) or acetaldehyde (10 ppm) which corresponds to the odor intensity of 5 based on the 6-grade odor intensity indicating method, and a gas containing methyl mercaptan (4 ppm) ch corresponds to 20-fold of the odor intensity of 5 were ventilated. After ventilation, a gas detecting tube (a gas detecting tube for ammonia: No. 3 L, a gas detecting tube for acetic acid: No. 81 L, a gas detecting tube for acetaldehyde: No. 92 L, a gas detecting tube for methyl mercaptan: No. 70 L) corresponding to the respective malodorous components was used to measure concentrations of the respective malodorous components in the passing gas. And the malodorous component reducing ratio was obtained by the following equation.

Malodorous component reducing ratio=[Malodorous component concentration beforeventilation-Malodorous component concentration after ventilation)/Malodorous component concentration before ventilation]×100

Deodorants including a chemical adsorption type deodorant in the following

Examples and Comparative Examples are shown in Table 1. Testing method for calculating a deodorization capacity of the respective deodorants is as shown below.

In a Tedlar® bag was charged 0.01 g of a deodorant, and after sealing, 2 L of a gas containing ammonia (8,000 ppm), methyl mercaptan (40 ppm), acetic acid (380 ppm) or acetaldehyde (2,000 ppm) which corresponds to 200-fold of a concentration of the odor intensity of 5 was filled therein. After 24 hours, concentrations (remaining gas component concentrations) of the respective malodorous components were measured with a gas detecting tube and the deodorization capacity (mL/g) was obtained by the following equation.

Deodorization capacity (mL/g)=[2000 (mL)×(Initial malodorous gas component concentration (ppm)-Remaining gas component concentration (ppm))×10⁻⁶]/0.01(g)

TABLE 1

| | | Deodorization | Average |
|---|---|---|---|
| | Target malodorous | capacity | diameter |
| Deodorant | component | (mL/g) | (μm) |
| Zirconium phosphate (Zr(HPO₄)₂•H₂O) | Ammonia | 150 | 0.8 |
| Aluminum silicate | Ammonia | 34 | 12 |
| CuO•SiO₂ composite oxide | Methyl mercaptan | 50 | 3 |
| Active zinc oxide | Acetic acid | 28 | 14 |
| Hydrous zirconium oxide | Acetic acid | 32 | 1 |
| Hydrotalcite | Acetic acid | 48 | 5 |
| 30% Adipic acid dihydrazide-carried silica gel | Acetaldehyde | 38 | 5 |
| Amorphous zeolite | Ammonia | 53 | 4 |
| Activated charcoal | Ammonia | 10 | 3 |

In addition, as a base material for the deodorizing filter produced in the following Examples and Comparative Examples, a nonwoven fabric sheet 1 in which a nonwoven fabric containing a polypropylene resin, a polyethylene resin and a polyethylene terephthalate resin is subjected to an interlacing treatment by needle-punching method or a nonwoven fabric sheet 2 in which a nonwoven fabric containing a polypropylene resin and a pol e h lene resin is produced by thermal bonding method was used.

### Example 1

### Production and Evaluation of Deodorizing Filter F1

A deodorant consisting of zirconium phosphate and a CuO.SiO₂ composite material shown in Table 1, and the nonwoven fabric sheet 1 were used. On the other hand, to develop the deodorant, a deodorant-containing processing liquid W1 having a solid concentration of 10% was prepared by using a zirconium phosphate powder, a CuO.SO₂ composite material powder and a polyester-based binder dispersion so that the mass ratio became 6 parts of zirconium phosphate, 6 parts of the CuO.SiO₂ composite material and 6 parts of the resin solid component of the polyester-based binder. The deodorant-containing processing liquid W1 was uniformly coated. onto the nonwoven fabric sheet 1 so that a spread amount of zirconium phosphate became 6 g/m² and a spread amount of the CuO.SiO₂ composite material became 6 g/m². After that, drying was conducted to prepare a deodorizing filter F1 in which the deodorant was uniformly adhered from one surface side to the other surface side. Then, a malodorous component reducing ratio, a basis weight, a thickness and permeability of the deodorizing filter F1 were measured, and the results were described in Table 2.

### Example 2

### Production and Evaluation of Deodorizing Filter F2

The deodorant-containing processing liquid W1 shown in Example 1 was uniformly coated onto the nonwoven fabric sheet 2 so that a spread amount of zirconium phosphate became 3 g/m² and a spread amount of the CuO.SiO₂ composite material became 3 g/m². After that, drying was conducted to prepare a deodorizing filter in which the deodorant was uniformly adhered from one surface side to the other surface side. Two sheets of the above deodorizing filters were prepared and laminated to prepare a deodorizing filter F2. Then, a malodorous component reducing ratio, a basis weight, a thickness and permeability of the deodorizing filter F2 were measured, and the results were described in Table 2.

### Example 3

### Production and Evaluation of Deodorizing Filter

The deodorant-containing processing liquid W1 shown in Example 1 was uniformly coated onto a nonwoven fabric sheet 1 having a different basis weight and thickness from those of Examples 1 and 2 so that a spread amount of zirconium phosphate became 3 g/m² and a spread amount of the CuO.SiO₂ composite material became 3 g/m². After that, drying was conducted to prepare a deodorizing filter F3 in which the deodorant was uniformly adhered from one surface side to the other surface side. Then, a malodorous component reducing ratio, a basis weight, a thickness and permeability of the deodorizing filter F3 were measured, and the results were described in Table 2.

### Example 4

### Production and Evaluation of Deodorizing Filter F4

The deodorantscontaining processing liquid W1 shown in Example 1 was uniformly coated onto a nonwoven fabric sheet 1 having a different basis weight and thickness from those of Examples 1 to 3 so that a spread amount of zirconium phosphate became 8 g/m² and a spread amount of the CuO.SiO₂ composite material became 8 g/m². After that, drying was conducted to prepare a deodorizing filter F4 in which the deodorant was uniformly adhered from one surface side to the other surface side. Then, a malodorous component reducing ratio, a basis weight, a thickness and permeability of the deodorizing filter F4 were measured, and the results were described in Table 2.

### Example 5

### Production and Evaluation of Deodorizing Filter F5

A deodorant consisting of aluminum silicate and hydrous zirconium oxide shown in Table 1, and the nonwoven fabric sheet 1 were used. On the other hand, to develop the deodorant, a deodorant-containing processing liquid W2 having a solid concentration of 10% was prepared by using an aluminum silicate powder, a hydrous zirconium oxide powder and a polyester-based binder dispersion so that the mass ratio became 6 parts of aluminum silicate, 5 parts of hydrous zirconium oxide and 5.5 parts of the resin solid component of the polyester-based binder. The deodorant-containing processing liquid W2 was uniformly coated onto the nonwoven fabric sheet 1 so that a spread amount of the aluminum silicate became 6 g/m² and a spread amount of the hydrous zirconium oxide became 5 g/m². After that, drying was conducted to prepare a deodorizing filter F5 in which the deodorant was uniformly adhered from one surface side to the other surface side. Then, a malodorous component reducing ratio, a basis weight, a thickness and permeability of the deodorizing filter F5 were measured, and the results were described in Table 2.

### Example 6

### Production and Evaluation of Deodorizing Filter F6

A deodorant consisting of zirconium phosphate, a CuO.SO₂ composite material and a 30% adipic acid dihydrazide-carried silica gel shown in Table 1, and the nonwoven fabric sheet 1 were used. On the other hand, to develop the deodorant, a deodorant-containing processing liquid W3 having a solid concentration of 10% was prepared by using a zirconium phosphate powder, a CuO.SiO₂ composite material powder, a 30% adipic acid dihydrazide-carried silica gel powder and a polyester-based binder dispersion so that the mass ratio became 6 parts of zirconium phosphate, 6 parts of the CuO.SiO₂ composite material, 4 parts of the 30% adipic acid dihydrazide-carried silica gel and 8 parts of the resin solid component of the polyester-based binder. The deodorant-containing processing liquid W3 was uniformly coated onto the nonwoven fabric sheet 1 so that a spread amount of the zirconium phosphate became 6 g/m², a spread amount of the CuO.Si₂ composite material became 6 g/m² and a spread amount of the 30% adipic acid dihydrazide-carried silica gel became 4 g/m². After that, drying was conducted to prepare a deodorizing filter F6 in which the deodorant was uniformly adhered from one surface side to the other surface side. Then, a malodorous component reducing ratio, a basis weight, a thickness and permeability of the deodorizing filter F6 were measured, and the results were described in Table 2.

### Example 7

### Production and Evaluation of Deodorizing Filter F7

A deodorant consisting of aluminum silicate and active zinc oxide shown in Table 1 and the nonwoven fabric sheet 1 were used. On the other hand, to develop the deodorant, a deodorant-containing processing liquid W4 having a solid concentration of 10% was prepared by using an aluminum silicate powder, an active zinc oxide powder and a polyester-based binder dispersion so that the mass ratio became 6 parts of aluminum silicate, 5 parts of the active zinc oxide and 5.5 parts of the resin solid component of the polyester-based binder. The deodorant-containing processing liquid W4 was uniformly coated onto the nonwoven fabric sheet 1 so that a spread amount of aluminum silicate became 6 g/m² and a spread amount of the active zinc oxide became 5 g/m². After that, drying was conducted to prepare a deodorizing filter F7 in which the deodorant was uniformly adhered from one surface side to the other surface side. Then, a malodorous component reducing ratio, a basis weight, a thickness and permeability of the deodorizing filter F7 were measured, and the results were described in Table 2.

### Example 8

### Production and Evaluation of Deodorizing Filter F8

A deodorant consisting of hydrous zirconium oxide and 30% adipic acid dihydrazide-carried silica gel shown in Table 1 and the nonwoven fabric sheet 1 were used. On the other hand, to develop the deodorant, a deodorant-containing processing liquid W5 having a solid concentration of 10% was prepared by using a hydrous zirconium oxide powder, a 30% adipic acid dihydrazide-carried silica gel powder and a polyester-based binder dispersion so that the mass ratio became 5 parts of hydrous zirconium oxide, 4 parts of the 30% adipic acid dihydrazide-carried silica gel and 4.5 parts of the resin solid component of the polyester-based binder. The deodorant-containing processing liquid W5 was uniformly coated onto the nonwoven fabric sheet 1 so that a spread amount of hydrous zirconium oxide became 5 g/m² and a spread amount of the 30% adipic acid dihydraz de-carried silica gel became 4 g/m². After that, drying was conducted to prepare a deodorizing filter F8 in which the deodorant was uniformly adhered from one surface side to the other surface side, Then, a malodorous component reducing ratio, a basis weight, a thickness and permeability of the deodorizing filter F8 were measured, and the results were described in Table 2.

### Example 9

### Production and Evaluation of Deodorizing Filter F9

A deodorant consisting of amorphous zeolite and hydrotalcite shown in Table 1, and the nonwoven fabric sheet 1 were used. On the other hand, to develop the deodorant, a deodorant-containing processing liquid W6 having a solid concentration of 10% was prepared by using an amorphous zeolite powder, a hydrotalcite powder and a polyester-based binder dispersion so that the mass ratio became 6 parts of the amorphous zeolite, 5 parts of hydrotalcite and 5.5 parts of the resin solid component of the polyester-based binder. The deodorant-containing processing liquid W6 was uniformly coated onto the nonwoven fabric sheet 1 so that a spread amount of the amorphous zeolite became 6 g/m² and a spread amount of hydrotalcite became 5 g/m². After that,drying was conducted to prepare a deodorizing filter F9 in which the deodorant has been uniformly adhered from one surface side to the other surface side. Then, a malodorous component reducing ratio, a basis weight, a thickness and permeability of the deodorizing filter F9 were measured, and the results were described in Table 2.

### Example 10

### Production and Evaluation of Deodorizing Filter F10

A deodorant consisting of zirconium phosphate, a CuO.SiO₂ composite material and hydrous zirconium oxide shown in Table 1, and the nonwoven fabric sheet 1 were used. On the other hand, to develop the deodorant, a deodorant-containing processing liquid W7 having a solid concentration of 10% was prepared by using a zirconium phosphate powder, a CuO.SiO₂ composite material powder, a hydrous zirconium oxide powder and a polyester-based binder dispersion so that the mass ratio became 6 parts of zirconium phosphate, 6 parts of the CuO.SiO₂ composite material, 5 parts of hydrous zirconium oxide and 8.5 parts of the resin solid component of the polyester-based binder. The deodorant-containing processing liquid W7 was uniformly coated onto the nonwoven fabric sheet 1 so that a spread amount of zirconium phosphate became 6 g/m², a spread amount of the CuO.SiO₂ composite material became 6 g/m² and a spread amount of hydrous zirconium oxide became 5 g/m². After that, drying was conducted to prepare a deodorizing filter F10 in which the deodorant was uniformly adhered from one surface side to the other surface side. Then, a malodorous component reducing ratio, a basis weight, a thickness and permeability of the deodorizing filter F10 were measured, and the results were described in Table 2.

### Example 11

### Production and Evaluation of Deodorizing Filter F11

A deodorant consisting of aluminum silicate, active zinc oxide and 30% adipic acid dihydrazide-carried silica gel shown in Table 1 and the nonwoven fabric sheet 1 were used. On the other hand, to develop the deodorant, a deodorant-containing processing liquid W8 having a solid concentration of 10% was prepared by using an aluminum silicate powder, an active zinc oxide powder, a 30% adipic acid dihydrazide-carried silica gel powder and a polyester-based binder dispersion so that the mass ratio became 6 parts of aluminum silicate, 5 parts of the active zinc oxide, 4 parts of the 30% adipic acid dihydrazide-carried silica gel and 7.5 parts of the resin solid component of the polyester-based hinder. The deodorant-containing processing liquid W8 was uniformly coated onto the nonwoven fabric sheet 1 so that a spread amount of aluminum silicate became 6 g/m², a spread amount of the active zinc oxide became 5 g/m² and a spread amount of the 30% adipic acid dihydrazide-carried silica gel became 4 g/m². After that, drying was conducted to prepare a deodorizing filter F11 in which the deodorant was uniformly adhered from one surface side to the other surface side. Then, a malodorous component reducing ratio, a basis weight, a thickness and permeability of the deodorizing filter F11 were measured, and the results were described in Table 2.

### Comparative Example 1

### Production and Evaluation of Deodorizing Filter F21

The deodorant-containing processing liquid W1 shown in Example 1 was uniformly coated onto the nonwoven fabric sheet 1 having a different basis weight and thickness from those of Examples 1 to 4, so that a spread amount of zirconium phosphate became 6 g/m² and a spread amount of the CuO.SiO₂ composite material became 6 g/m². After that, drying was conducted to prepare a deodorizing filter F21 in which the deodorant was uniformly adhered from one surface side to the other surface side. Then, a malodorous component reducing ratio, a basis weight, a thickness and permeability of the deodorizing filter F21 were measured, and the results were described in Table 3.

### Comparative Example 2

### Production and Evaluation of Deodorizing Filter F22

The deodorant-containing processing liquid W1 shown in Example 1 was uniformly coated onto the nonwoven fabric sheet 1 having a different basis weight and thickness from those of Example 1 to 4 and Comparative Example 1, so that a spread amount of zirconium phosphate became 6 g/m² and a spread amount of the CuO.SiO₂ composite material became 6 g/m². After that, drying was conducted to prepare a deodorizing filter F22 in which the deodorant was uniformly adhered from one surface side to the other surface side. Then, a malodorous component reducing ratio, a basis weight, a thickness and permeability of the deodorizing filter F22 were measured, and the results were described in Table 3.

### Comparative Example 3

### Production and Evaluation of Deodorizing Filter F23

The deodorant-containing processing liquid W3 shown n Example 6 was uniformly coated onto the nonwoven fabric sheet 1 having a different basis weight and thickness from those of Example 6 so that a spread amount of zirconium phosphate became 6 g/m², a spread amount of the CuO.SiO₂ composite material became 6 g/m² and a spread amount of the 30% adipic acid dihydrazide-carried silica gel became 4 g/m². After that, drying was conducted to prepare a deodorizing filter F23 in which the deodorant was uniformly adhered from one surface side to the other surface side. Then, a malodorous component reducing ratio, a basis weight, a thickness and permeability of the deodorizing filter F23 were measured, and the results were described in Table 3.

### Comparative Example 4

### Production and Evaluation of Deodorizing Filter F24

The deodorant-containing processing liquid W3 shown in Example 6 was uniformly coated onto the nonwoven fabric sheet 1 having a different basis weight and thickness from those of Example 6 and Comparative Example 3, so that a spread amount of zirconium phosphate became 6 g/m², a spread amount of the CuO.SiO₂ composite material became 6 g/m² and a spread amount of the 30% adipic acid dihydrazide-carried silica gel became 4 g/m². After that, drying was conducted to prepare a deodorizing filter F24 in which the deodorant was uniformly adhered from one surface side to the other surface side. Then, a malodorous component reducing ratio, a basis weight, a thickness and permeability of the deodorizing filter F24 were measured, and the results were described in Table 3.

### Comparative Example 5

### Production and Evaluation of Deodorizing Filter F25

The deodorant-containing processing liquid W6 shown n Example 9 was uniformly coated onto the nonwoven fabric sheet 1, so that a spread amount of amorphous zeolite became 6 g/m² and a spread amount of hydrotalcite became 5 g/m². After that, drying was conducted to prepare a deodorizing filter F25 in which the deodorant was uniformly adhered from one surface side to the other surface side. Then, a malodorous component reducing ratio, a basis weight, a thickness and permeability of the deodorizing filter F25 were measured, and the results were described in Table 3.

### Comparative Example 6

### Production and Evaluation of Deodorizing Filter F26

An activated charcoal and the nonwoven fabric sheet 1 were used. On the other hand, to develop activated charcoal, a deodorant-containing processing liquid W9 having a solid concentration of 10% was prepared by using an activated charcoal powder and a polyester-based binder dispersion so that the mass ratio became 12 parts of the activated charcoal and 6 parts of the resin solid component of the polyester-based binder. The deodorant-containing processing liquid W9 was uniformly coated onto the nonwoven fabric sheet 1, so that a spread amount of activated charcoal became 12 g/m². After that, drying was conducted to prepare a deodorizing filter F26 in which the deodorant was uniformly adhered from one surface side to the other surface side. Then, a malodorous component reducing ratio, a basis weight, a thickness and permeability of the deodorizing filter F26 were measured, and the results were described in Table 3.

TABLE 2

| | | | | Deodorant- | Deodor- | | | Perme- | | Malodorous |
|---|---|---|---|---|---|---|---|---|---|---|
| | | Deodor- | | containing | ant | Basis | Thick- | ability | Target | component |
| | | izing | | processing | content | weight | ness | [cm³/ | malodorous | reducing ratio |
| | | filter | Deodorant | liquid | [g/m²] | [g/m²] | [mm] | (cm² · s)] | component | [%] |
| Example | 1 | F1 | Zirconium phosphate | W1 | 6 | 68 | 0.8 | 228 | Ammonia | 99 |
| | | | CuO•SiO₂ composite oxide | | 6 | | | | Methyl | 95 |
| | | | | | | | | | mercaptan | |
| | 2 | F2 | Zirconium phosphate | W1 | 6 | 78 | 0.6 | 53 | Ammonia | 99 |
| | | | CuO•SiO₂ composite oxide | | 6 | | | | Methyl | 97 |
| | | | | | | | | | mercaptan | |
| | 3 | F3 | Zirconium phosphate | W1 | 3 | 58 | 0.7 | 251 | Ammonia | 93 |
| | | | CuO•SiO₂ composite oxide | | 3 | | | | Methyl | 92 |
| | | | | | | | | | mercaptan | |
| | 4 | F4 | Zirconium phosphate | W1 | 8 | 84 | 0.8 | 184 | Ammonia | 99 |
| | | | CuO•SiO₂ composite oxide | | 8 | | | | Methyl | 98 |
| | | | | | | | | | mercaptan | |
| | 5 | F5 | Aluminum silicate | W2 | 6 | 72 | 0.7 | 180 | Ammonia | 92 |
| | | | Hydrous zirconium oxide | | 5 | | | | Acetic acid | 93 |
| | 6 | F6 | Zirconium phosphate | | 6 | | | | Ammonia | 92 |
| | | | CuO•SiO₂ composite oxide | W3 | 6 | 42 | 0.9 | 312 | Methyl | 91 |
| | | | | | | | | | mercaptan | |
| | | | 30% Adipic acid dihydrazide- | | 4 | | | | Acetaldehyde | >90 |
| | | | carried silica gel | | | | | | | |
| | 7 | F7 | Aluminum silicate | W4 | 6 | 65 | 0.9 | 250 | Ammonia | 92 |
| | | | Active zinc oxide | | 5 | | | | Acetic acid | 93 |
| | 8 | F8 | Hydrous zirconium oxide | W5 | 5 | 80 | 0.6 | 125 | Acetic acid | >93 |
| | | | 30% Adipic acid dihydrazide- | | 4 | | | | Acetaldehyde | >90 |
| | | | carried silica gel | | | | | | | |
| | 9 | F9 | Amorphous zeolite | W6 | 6 | 55 | 0.6 | 175 | Ammonia | 90 |
| | | | Hydrotalcite | | 5 | | | | Acetic acid | 91 |
| | 10 | F10 | Zirconium phosphate | W7 | 6 | 82 | 1.2 | 271 | Ammonia | 93 |
| | | | CuO•SiO₂ composite oxide | | 6 | | | | Methyl | 91 |
| | | | | | | | | | mercaptan | |
| | | | Hydrous zirconium oxide | | 5 | | | | Acetic acid | 92 |
| | 11 | F11 | Aluminum silicate | W8 | 6 | 73 | 1.0 | 256 | Ammonia | 93 |
| | | | Active zinc oxide | | 5 | | | | Acetic acid | 92 |
| | | | 30% Adipic acid dihydrazide- | | 4 | | | | Acetaldehyde | >90 |
| | | | carried silica gel | | | | | | | |

TABLE 3

| | | | | Deodorant- | Deodor- | | | Perme- | | Malodorous |
|---|---|---|---|---|---|---|---|---|---|---|
| | | Deodor- | | containing | ant | Basis | Thick- | ability | Target | component |
| | | izing | | processing | content | weight | ness | [cm³/ | malodorous | reducing ratio |
| | | filter | Deodorant | liquid | [g/m²] | [g/m²] | [mm] | (cm² · s)] | component | [%] |
| Comparative | 1 | F21 | Zirconium phosphate | W1 | 6 | 78 | 1.2 | 412 | Ammonia | 55 |
| Example | | | CuO•SiO₂ composite oxide | | 6 | | | | Methyl | 35 |
| | | | | | | | | | mercaptan | |
| | 2 | F22 | Zirconium phosphate | W1 | 6 | 70 | 0.4 | 39 | Ammonia | 78 |
| | | | CuO•SiO₂ composite oxide | | 6 | | | | Methyl | 75 |
| | | | | | | | | | mercaptan | |
| | 3 | F23 | Zirconium phosphate | W3 | 6 | 56 | 0.3 | 155 | Ammonia | 60 |
| | | | CuO•SiO₂ composite oxide | | 6 | | | | Methyl | 45 |
| | | | | | | | | | mercaptan | |
| | | | 30% Adipic acid dihydrazide- | | 4 | | | | Acetaldehyde | 55 |
| | | | carried silica gel | | | | | | | |
| | 4 | F24 | Zirconium phosphate | W3 | 6 | 110 | 0.5 | 19 | Ammonia | 95 |
| | | | CuO•SiO₂ composite oxide | | 6 | | | | Methyl | 86 |
| | | | | | | | | | mercaptan | |
| | | | 30% Adipic acid dihydrazide- | | 4 | | | | Acetaldehyde | 79 |
| | | | carried silica gel | | | | | | | |
| | 5 | F25 | Amorphous zeolite | W6 | 6 | 29 | 0.5 | 450 | Ammonia | 31 |
| | | | Hydrotalcite | | 5 | | | | Acetic acid | 29 |
| | 6 | F26 | Activated charcoal | W9 | 12 | 65 | 0.8 | 230 | Ammonia | 45 |

From the results in Table 2 and Table 3, the following can be understood. All of Examples 1 to 11 showed high deodorizing performances with the malodorous component reducing ratio of 90% or higher. On the other hand, Comparative Example 1 is an example in which the permeability of the deodorizing filter is too high, so that deodorizing performances are poor. Comparative Example 2 is an example in which the permeability of the deodorizing filter is too low and a thickness of the deodorizing fiber layer (the deodorizing filter) is too thin, so that deodorizing performances are poor. Comparative Example 3 is an example in which the thickness of the deodorizing fiber layer (the deodorizing filter) is too thin, so that deodorizing performances are poor. Comparative Example 4 is an example in which the basis weight of the deodorizing filter is too high and permeability is too low,so that deodorizing performances are not sufficient and it does not act as a filter since the permeability is too low. Comparative Example 5 is an example in which the basis weight of the deodorizing filter is too low and the permeability is too high, so that deodorizing performances are poor. Comparative Example 6 is an example of a deodorizing filter in which the physical adsorption type deodorant is processed in place of the chemical adsorption type deodorant, so that deodorizing performances are poor. Accordingly, to obtain high deodorizing performances, it is necessary that a chemical adsorption type deodorant is used in a deodorizing fiber layer having a specific thickness and basis weight, and a deodorizing filter has a specific permeability.

In the following Example 12 and Comparative Example 7, a deodorization effect persistence of the deodorizing filter was evaluated using 10 ppm of a methyl mercaptan gas.

### Example 12

To the deodorizing filter F1 produced in Example 1 was ventilated the methyl mercaptan gas at two-minute intervals, and the malodorous component reducing ratio at each time after the ventilation was calculated in the manner as mentioned above to evaluate the deodorization effect persistence. The results were shown in FIG. 1.

### Comparative Example 7

Evaluation of the deodorizing filter F26 produced in Comparative Example 6 in place of the deodorizing ter Flwas conducted in the same manner as those in Example 12. The results were shown in FIG. 1.

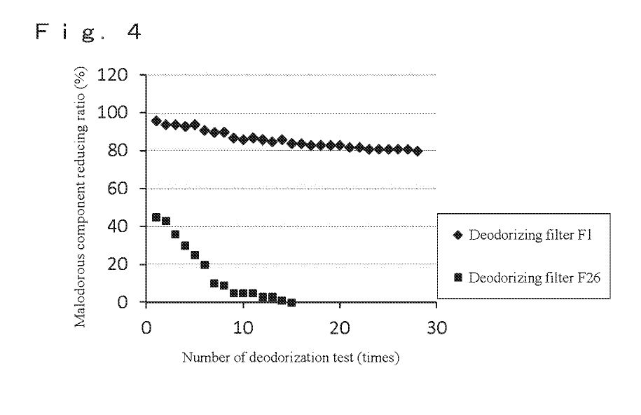

As clearly seen from FIG. 4, the malodorous component reducing ratio in Comparative Example 7 using the deodorizing filter F26 became 0% after 15 times of the repeating tests, while the malodorous component reducing ratio in Example 12 using the deodorizing filter F1 was maintained at 80% or higher till 28 times of the repeating tests, and showed high persistence of the deodorization effect.

### INDUSTRIAL APPLICABILITY

According to the deodorizing filter of the present invention, high deodorizing performances can be instantaneously obtained to a malodorous gas passing through the deodorizing filter in an atmosphere containing a stool odor, a putrid odor, or the like. Consequently, the deodorizing filter is useful as a filter for a mask, or a filter for an air cleaner, an air conditioner and the like to avoid bad smell generated in medical, caregiving and excrementitious fields, a wastewater treatment plant, a refuse treatment plant (an incineration plant), a fertilizer factory, a chemical factory, or the like; an animal smell, a stool odor, a putrid odor (including a bad odor from a pet or an article for pets) generated in a livestock farm, a fishing port, an animal-related institution, or the like; bad smell from a foot stepping mat, shoe insoles, a shoe cupboard, a trash can, a toilet, or the like.

### REFERENCE SIGNS LIST

1: deodorizing filter, 10: deodorizing fiber layer, 11: fiber, 13: chemical adsorption type deodorant, 15: joint portion (binder resin)

1. A deodorizing filter comprising a deodorizing fiber layer that includes a fiber and a chemical adsorption deodorant ed to a surface of the fiber, wherein:
a thickness of the deodorizing fiber layer is 0.3 mm or more;
a basis weight of the deodorizing fiber layer ranges from 30 to 100 g/m²; and
a permeability of the deodorizing filter measured by fragile form method ranges from 50 to 350 cm³/(cm²·s).

2. The deodorizing filter according to claim 1, wherein a base material of the deodorizing filter is a nonwoven fabric.

3. The deodorizing filter according to claim 1, wherein the chemical adsorption deodorant is (1) a tetravalent metal phosphate, (2) an amine compound, (3) a zeolite, (4) an amorphous composite oxide represented by X₂O—Al₂O₃—SiO₂ (X is at least one atom selected from Na, K and Li), (5) a composite material containing at least one atom selected from Ag, Cu, Zn and Mn, (6) at least one zirconium compound selected from hydrated zirconium oxide and zirconium oxide, (7) a hydrotalcite-based compound, or (8) an amorphous active oxide.

4. The deodorizing filter according to claim 1, wherein a content of the chemical adsorption deodorant ranges from 2 to 60 parts by mass based on 100 parts by mass of the fiber constituting the deodorizing fiber ayer.

5. The deodorizing filter according to claim 1, wherein a median diameter measured by a laser diffraction particle size distribution analyzer of the chemical adsorption deodorant ranges from 0.05 to 100 μm.

6. The deodorizing filter according to claim 1, wherein the chemical adsorption deodorant is joined to the fiber by a binder resin in the deodorizing fiber layer.

7. The deodorizing filter according to claim 6, wherein a content atio of the binder resin and the chemical adsorption deodorant is respectively 10 to 90% by' mass and 10 to 90% by mass based on 100% by mass of a total of the binder resin and the chemical adsorption deodorant.
